(19) United States
(12) Patent Application Publication
(10) Pub. No.: US 2023/0248897 A1
(43) Pub. Date: Aug. 10, 2023

(54) IMPACT RESISTANT AND TAMPER EVIDENT SYSTEM FOR PREFILLED SYRINGE

(71) Applicant: FRESENIUS KABI USA, LLC, Lake Zurich, IL (US)

(72) Inventor: Christopher J. Murray, Chicago, IL (US)

(21) Appl. No.: 18/136,054

(22) Filed: Apr. 18, 2023

Related U.S. Application Data
(63) Continuation-in-part of application No. 16/949,226, filed on Oct. 20, 2020.

(51) Int. Cl. A61M 5/00; A61M 5/32

(52) U.S. Cl. CPC A61M 5/002; A61M 5/3202; A61M 2205/02; A61M 2205/19

(57) ABSTRACT

A package includes a tray having a plurality of openings and a plurality of barrel covers. Each of the plurality of barrel covers extend through one of the plurality of openings. A tub can receive the tray. A plurality of tamper evident devices can be attached to the plurality of barrel covers. Each tamper evident device includes a collar, a tamper evident cap, and a frangible connection between the collar and the tamper evident cap. A method of packaging a plurality of syringe bodies includes inserting the plurality of syringe bodies into a plurality of barrel covers positioned in a tray, and inserting the tray into a tub.

### PRIORITY

This patent application is a continuation-in-part of U.S. patent application Ser. No. 16/949,226, filed on Oct. 20, 2020, the disclosure of which is incorporated herein in its entirety.

### TECHNICAL FIELD

This disclosure relates generally to devices and methods of manufacturing, packaging, and using syringes containing medical materials, and more particularly relates to an impact resistant and tamper evident system for a prefilled syringe.

### BACKGROUND

Syringe assemblies are used to hold, transport, and deliver materials. For example, syringes are often utilized in medical environments to administer one or more medicinal components to a patient. Syringes can be delivered to a user empty or prefilled Empty syringes are intended to be filled by the user from a vial or other container, whereas prefilled syringes are supplied to the user with a material (e.g., medicament or diluent) provided therein by a manufacturer. When prefilled, the syringes are often filled at a manufacturer site and shipped to the user ready-to-use.

### SUMMARY

Manufacturers have a number of difficulties in effectively producing, packaging, and shipping the prefilled syringes. One issue is that the syringes can be fragile and often break during the shipping and handling. Another issue is that the manufacturer needs to ensure that the material in the syringe has not been tampered with after filling. Yet another issue is that the internal fluid pressure of the prefilled syringe can lead to the plunger rod popping out of the syringe during shipping. Therefore, there is a need for improved syringe assemblies and methods of assembling prefilled syringes.

The foregoing needs are met by the various embodiments of packages disclosed.

One aspect of a package may include a tray having a plurality of openings, and a plurality of barrel covers, where each of the plurality of barrel covers extend through one of the plurality of openings.

Another aspect of a package includes a tray having a plurality of tube members extending from an upper surface, wherein each of the plurality of tube members defines an opening. The package also includes a tub receiving the tray, a plurality of barrel covers extending through the openings of the plurality of tube members, a plurality of syringe bodies in the plurality of barrel covers, and a plurality of tamper evident devices attached to the plurality of barrel covers. Each tamper evident device includes a collar, a tamper evident cap, and a frangible connection between the collar and the tamper evident cap.

Another aspect of the present disclosure is directed to a method of packaging. The method may include inserting the plurality of syringe bodies into a plurality of barrel covers positioned in a tray, and inserting the tray into a tub. In some aspects, the method may include inserting the plurality of barrel covers into a plurality of opening of the tray. In some aspects, the method may include sealing the tub by attaching a sheet to an upper flange of the tub, where the sheet is permeable to sterilizing gas and/or vapor. In some aspects, the method may include releasably attaching the plurality of barrel covers to the tray via at least one of a friction fit, a press fit, a snap fit, a rotational connection, or a frangible connection.

In some aspects, the barrel cover is translucent or transparent. In some aspects, the plurality of syringe bodies are empty. In some aspects, the package includes a plurality of syringe bodies, where each of the syringe bodies is inserted into one of the plurality of barrel covers through a corresponding opening. In some aspects, the package includes a plurality of syringe caps, where each of the plurality of syringe caps are configured to seal a distal portion of one of the plurality of syringe bodies when the plurality of syringe bodies are inserted into the plurality of barrel covers through the plurality of openings. In some aspects, the package includes a plurality of the tamper evident devices including the tamper evident cap, the collar, and the frangible connection between the tamper evident cap and the collar.

In some aspects, each of the plurality of syringe bodies includes a flange at a proximal portion of the syringe body, and each of the plurality of barrel covers includes a plurality of ridges supporting the flange of one of the plurality of syringe bodies. In some aspects, each of the plurality of barrel covers has a proximal opening and a distal opening, and each of the plurality of syringe bodies extends distal of the distal opening of each of the plurality of barrel covers. In some aspects, the package may include a plurality of tamper evident devices, where each collar of each of the plurality of tamper evident devices is attached to one of the plurality of barrel covers. In some aspects, the tray may have a plurality of tube members extending from an upper surface of the tray, each of the plurality of tube members defining one of the plurality of openings, and each of the plurality of barrel covers being supported by one of the plurality of tube members.

In some aspects, the package includes a tub, wherein the tub includes a shoulder portion configured to support the tray. In some embodiments, the package further includes a sheet adhered to a rim of the tub and covering the tray, wherein the sheet is permeable to sterilizing gas and/or vapor. In some embodiments, the barrel covers are releasably attached and/or secured to the tray by one or more of a friction fit, a press fit, a snap fit, a rotational connection, and/or a frangible connection.

### BRIEF DESCRIPTION OF THE DRAWINGS

The present application is further understood when read in conjunction with the appended drawings. For the purpose of illustrating the subject matter, there are shown in the drawings exemplary embodiments of the subject matter; however, the presently disclosed subject matter is not limited to the specific methods, devices, and systems disclosed. In the drawings:

FIG. 1 illustrates an isometric view of an embodiment of a syringe assembly.

FIG. 2 illustrates an isometric view of a barrel cover containing a syringe body.

FIG. 3 illustrates a proximal view of the barrel cover.

FIG. 4 illustrates an isometric view of a tamper evident device.

FIGS. 5A-C illustrate exploded views of embodiments of the tamper evident device and the barrel cover.

FIG. 6 illustrates an isometric view of a released configuration of the tamper evident device.

FIG. 7 illustrates an exploded view of a tip assembly.

FIG. 8 illustrates a partially exploded view of the syringe assembly.

FIG. 9 illustrates an exploded view of the syringe body and the barrel cover stored in a container of a package.

FIG. 10 illustrates a side view of the package of a plurality of the syringe bodies and the barrel covers in the container that is sealed.

FIGS. 11A-C illustrate embodiments of the package where the barrel covers are releasably attached and/or secured to a tray of the package.

FIG. 12 illustrates a flow chart of a method of packaging the syringe bodies.

FIG. 13 illustrates a flow chart of a method of filling the syringe bodies and packaging the syringe assemblies as prefilled syringe assemblies.

Aspects of the disclosure will now be described in detail with reference to the drawings, wherein like reference numbers refer to like elements throughout, unless specified otherwise.

### DETAILED DESCRIPTION OF ILLUSTRATIVE EMBODIMENTS

An impact resistant and tamper evident system for a prefilled syringe, and method of assembling the system, are described. The system may provide a protective enclosure during shipping, handling, storage, and/or use of the prefilled syringe, without requiring secondary packaging. The system may have a barrel cover that protects the syringe body and is translucent and/or transparent to allow the clinician to view labels and/or graduation marks in or on the syringe body. The barrel cover may, additionally or alternatively, protect the material from environmental factors that may negatively impact biological stability of the material. The system may further have backstop members that snap on a proximal portion of the barrel cover to secure and prevent a plunger rod from being removed or fluid from being diverted through a stopper. The system may further have a tamper evident device disposed over a distal tip of the syringe body and at a distal portion of the barrel cover. The tamper evident device may include a tamper evident cap removable from the distal tip of the syringe body by a user twisting and breaking frangible bridges. An anti-rotation feature on the barrel cover and/or tamper evident device may prevent rotation of the remaining portion of the tamper evident device during removal of the cap.

The impact resistant and tamper evident system of the present disclosure provides a number of advantageous features that benefit the manufacturer and user, as discussed herein. The system ensures protection of the syringe and filled material, while allowing the user to administer the material without removal of the syringe from the barrel cover. The system prevents the plunger rod from being removed from the syringe chamber ensuring containment of the material. The system mechanically protects the syringe during shipping by providing a protective cover and reducing movement of the syringe relative to the protective barrel cover. The system also allows the manufacturer and user to view the medicament and/or indicia in or on the syringe, while the syringe is protected by the barrel cover.

The syringe body may be inserted, when empty, into the barrel cover and packaged into a container, such that the barrel cover protects the syringe body prior to filling the syringe with a material. The packaged container may include a tub having an opening, a nest inserted into the opening, and a sheet sealing the opening. The tamper evident device may be assembled to the barrel cover prior to insertion of the syringe body, in order to further increase assembly efficiency by reducing processing time and steps needed to be performed after filling of the syringe body. For example, in some embodiments, a plurality of barrel covers optionally with tamper evident devices attached thereto may be inserted into the tray to reduce the number of steps of packaging, filling, and assembling the prefilled syringes. The package including a plurality of the empty syringe bodies contained with a plurality of barrel covers optionally with tamper evident devices attached thereto may be sterilized and transported to a fill station, where the package may be unsealed, and the syringe bodies may be filled and sealed with the stopper. The plunger rod and/or the backstop may be assembled to the syringe body and repackaged as a prefilled syringe assembly. Additional advantageous features of the impact resistant and tamper evident system are evident from the present disclosure.

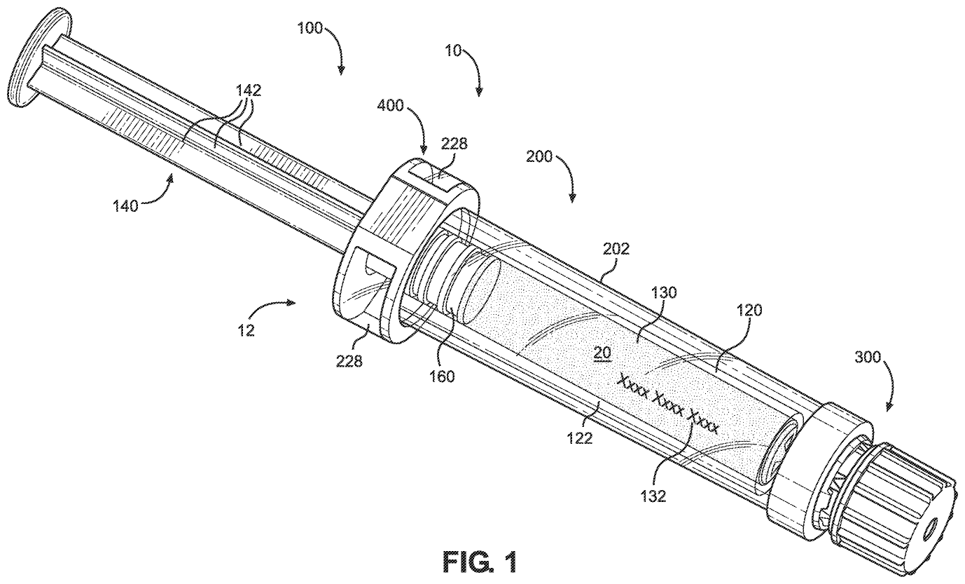

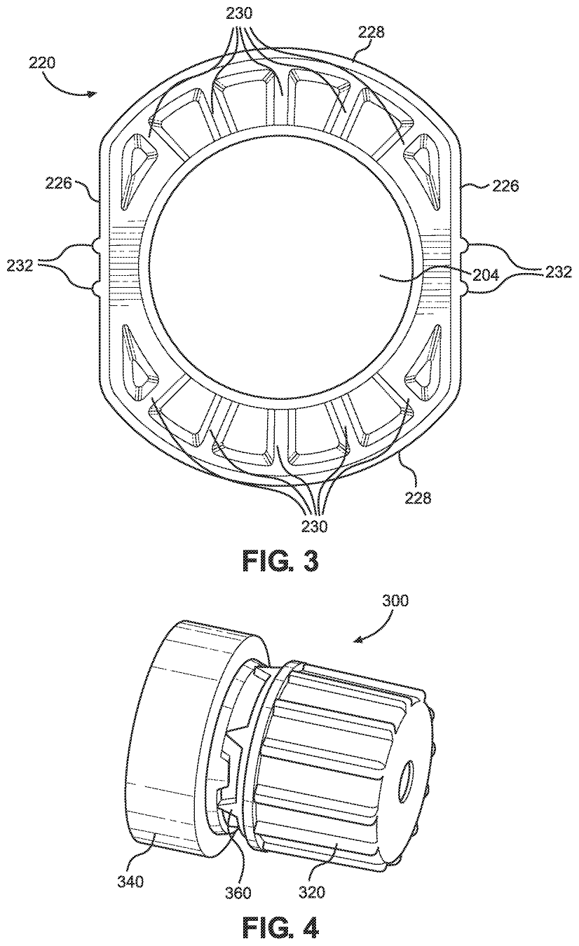

FIGS. 1-8 illustrate exemplary embodiments of a syringe assembly 10 containing a material 20, such as a liquid medicament. The syringe assembly 10 may have a flange 12 and include a syringe 100, a barrel cover 200, a tamper evidence device 300, and a backstop 400.

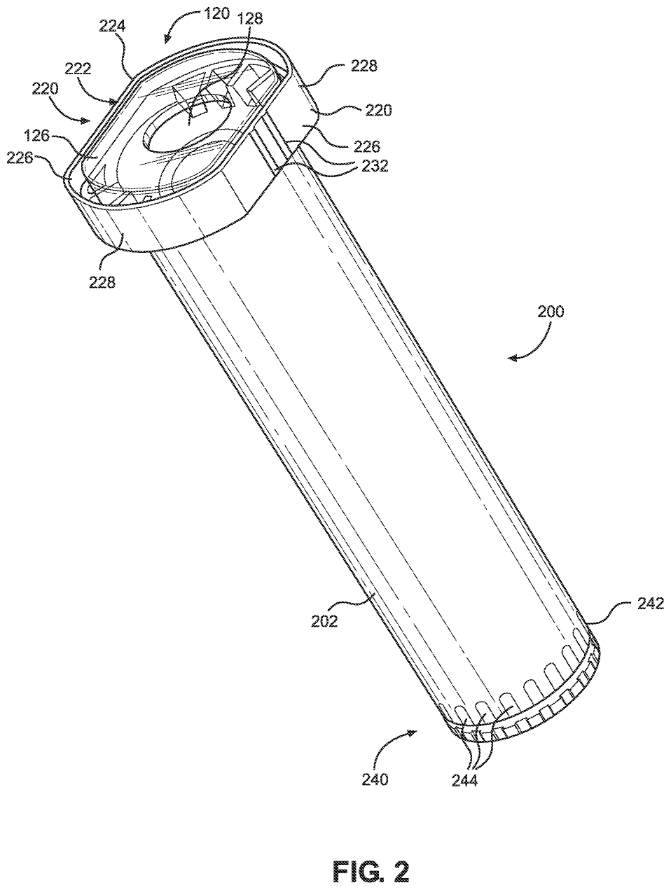

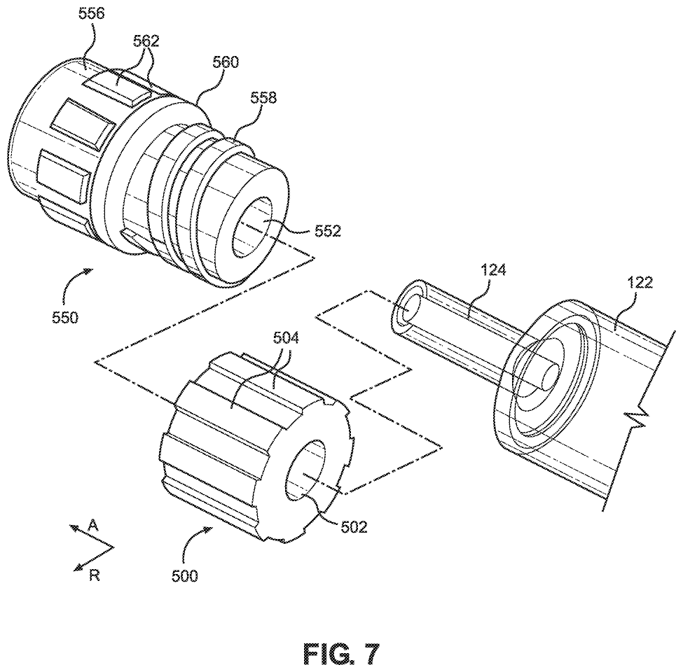

The syringe 100 may include a syringe body 120 and a plunger rod 140. The syringe body 120 may have a syringe barrel 122 extending from a proximal end to a distal end along a longitudinal direction. The syringe body 120 may further have a syringe tip 124 at the distal end (as illustrated in FIG. 7) and a syringe flange 126 at the proximal end (as illustrated in FIG. 2). The syringe barrel 122 may be substantially cylindrical having an inner surface extending along the longitudinal direction to define a chamber 130. The chamber 130 may be configured to receive, store, and/or mix the material 20 for dispensing through a distal opening of the syringe tip 124. The syringe body 120 may have indicia 132 in and/or on an outer surface of the syringe barrel 122 providing data and/or information of the material 20, such as name, dose, expiration date, instructions, and/or recipient. The indicia 132 may, additionally or alternatively, include graduations providing measurements of the remaining amount of the material 20. The indicia 132 may, additionally or alternatively, include a marking representing data in machine-readable form, such as a linear barcode, radio frequency identification tag, and/or QR code. In some embodiments, the indicia 132 are provided on a label that is affixed to the outer surface of the syringe barrel 122.

The syringe tip 124 may include a connection interface for engagement with an external device (not shown), such as a syringe needle, a medical transport line, or a container. The syringe tip 124 may be tapered and/or further have a Luer connection. The syringe flange 126 may extend radially outwards from the proximal end of the syringe body 120 to enable stability and handling of the syringe body 120. The syringe flange 126 may have a substantially oval shape with two flats, oppositely positioned sides and two arcuate, oppositely positioned sides (as illustrated FIG. 2). Alternatively, the syringe flange 126 may have a different shape, such as a substantially circular shape. The syringe flange 126 may have a proximal opening 128 sized to receive the plunger rod 140 and defining a proximal end of the chamber 130.

The plunger rod 140 may have a plunger stopper 160 at a distal end to define the proximal-most extent to which the material 20 can fill the chamber 130. The plunger stopper 160 may have an enlarged, substantially cylindrical body. The plunger stopper 160 may be made of a flexible elastomeric material (e.g., rubber) and generally conform to the shape of the chamber 130 to prevent leakage of the material 20 proximally out of the chamber 130. As the plunger stopper 160 moves distally through the chamber 130, the plunger stopper 160 may push the material 20 out of the chamber 130 through the syringe tip 124. Similarly, as the plunger stopper 16A) moves proximally through the chamber 130, the plunger stopper 160 may create a vacuum to draw the material 20 into the chamber 130 through the syringe tip 124. The plunger rod 140 may include a plurality of vanes 142 extending radially and longitudinally along the length of the plunger rod 140. The plunger rod 140 may include four vanes 142, where the vanes 142 are arranged circumferentially spaced apart 90 degrees forming a substantially plus-shaped orientation. The vanes 142 may function to provide stability and strength to the plunger rod 140, while minimizing the cross-sectional footprint of the plunger rod 140 to reduce material requirements for the plunger rod 140, thus reducing overall weight of the syringe assembly.

The syringe body 120 and/or plunger rod 140 may be made of glass or a translucent and/or transparent plastic material, such as polyethylene terephthalate (PET), polypropylene (PP), polycarbonate (PC), or other material. However, the invention may be particularly suitable for embodiments where the syringe body 120 is made of glass, which provides a stable container reducing biological degradation of the material 20 and extending the expiration.

As further illustrated in FIGS. 1-3, the syringe body 120 may be received in the barrel cover 200 to provide a protective enclosure without requiring secondary packaging. The barrel cover 200 may include a tubular barrel 202 extending from a proximal end to a distal end along a longitudinal direction. The barrel cover 200 may have a proximal opening 204 to receive the syringe body 120 and a distal opening 206 to receive the syringe tip 124 when the syringe body 120 is inserted. The distal opening 206 may allow the syringe 100 to dispense the material 20 while inside the barrel cover 200 to ensure protection.

The barrel cover 200 may be made of an impact resistant, translucent and/or transparent plastic material, such as polyethylene terephthalate (PET), polypropylene (PP), polycarbonate (PC), or other material. The barrel cover 200 made of plastic may provide mechanical protection to the syringe body 120 against external forces during shipping, handling, storage, and/or use of the syringe 100, especially useful when the syringe body 120 is made of glass. At least a portion of the barrel cover 200 (e.g., the entire length of the tubular barrel 202) may form a viewing window that allows the user to see the indicia 132 in and/or on the outer surface of the syringe barrel 122. Thus, the barrel cover 200 may provide visual access to essential data/information of the syringe 100, while maintaining protection of the syringe 100. The barrel cover 200 may also provide visual access to the contents of the syringe 100, to determine the volume and/or status of the material 20 remaining in the syringe 100. In some embodiments, the color, transparency and/or other attributes of the barrel cover 200 may be selected based on, for example, a characteristic, such as the UV sensitivity, of the material 20 contained inside the syringe. Additionally, the color and/or other attributes of the barrel cover 20) may be selected based on a desired color-coding scheme of the syringe packaging and/or other labeling considerations. In some embodiments, the barrel cover 200 may have indicia in and/or on an outer surface of the barrel cover 200 providing data and/or information of the material 20, such as name, dose, expiration date, instructions, and/or recipient in human and/or machine-readable form. In certain embodiments, the indicia are provided on a label that is affixed to the outer surface of the barrel cover 200.

The barrel cover 200 may further have a flange 220 at the proximal end and an anti-rotation feature 240 at the distal end. The anti-rotation feature 240 may include an annular groove 242 and a plurality of teeth or grooves 244, according to some embodiments. The teeth or grooves 244 may be longitudinally interrupted by the annular groove 242, such that two sets of teeth or grooves 244 are formed on opposing longitudinal sides of the annular groove 242. The cover flange 220 may have a recess 222 defined by a circumferential wall 224 and configured to receive the syringe flange 126 of the syringe barrel 122. The cover flange 220 may have a substantially oval shape including first and second opposing substantially flat wall portions 226 and first and second opposing arcuate wall portions 228. Alternatively, the cover flange 220 may have a different shape, such as a substantially circular shape. The cover flange 220 may be slightly larger than the syringe flange 126 and have a shape that substantially matches the shape of the syringe flange 126, such that the circumferential wall 224 surrounds the syringe flange 126. The cover flange 220 may include a plurality of ridges 230 extending proximally and radially around/from the proximal opening 204 and positioned below the proximal end of the circumferential wall 224. The ridges 230 may be configured to support the syringe flange 126 providing stability and an improved seat for the syringe flange 126. The ridges 230 may also improve manufacturability, for example, by reducing deformation and ease of ejection of the syringe body 120 from a molding tool. The cover flange 220 may additionally include ridges and/or grooves 232 configured to secure the backstop members 402.

As further illustrated in FIGS. 4-6, the tamper evident device 300 may include a cap 320, a collar 340, and a frangible connection 360, each defining a longitudinal passage 302. The tamper evident device 300 may ensure that the material 20 is not improperly accessed after filling, during shipping, handling, and/or storage.

The tamper evident cap 320 may be a substantially hollow cylinder and define an outer surface and an inner surface. The inner surface may define the passage 302 configured to receive the syringe tip 124 and/or a tip cap 550. The tamper evident cap 320 may have an open proximal end 324 and a closed distal end 326, where the passage 302 extends from the open proximal end 324 along the longitudinal direction and terminates within the main body at a location proximal to the distal end 326. However, in other embodiments, the distal end of the tamper evident cap 320 may be open or partially closed.

The collar 340 may be initially attached to the cap 320 and substantially surround a distal portion of the barrel cover 200. The collar 340 may include an annular rib 342 in an inner surface configured to snap into the annular groove 242 on the outer surface of the barrel cover 200 during assembly to longitudinally fix the collar 340 and cap 320. The snap connection between the collar 340 and barrel cover 200 may provide a tactile and/or audible indication of a proper connection.

The frangible connection 360 may include a plurality of frangible bridges 362 spaced around the circumference of the tamper evident device 300. The frangible bridged 362 may releasably connect the tamper evident cap 320 and the collar 340, such that when the frangible connection 360 is broken, the tamper evident cap 320 may be removed from the syringe tip 124. Each of the frangible bridges 362 may embody a thin portion of the tamper evident device 300 that tapers inwards in width as it extends proximally. However, the frangible bridges 362 may be alternatively configured, such as having a constant width. Further, the frangible bridges 362 may be equidistantly spaced about the circumference of the tamper evident device 300. The frangible connection 360 may define a plurality of gaps between the frangible bridges 362, which allow the tamper evident device 300 to be broken at the frangible connection 360 by the user providing a substantial rotational or twisting force to the tamper evident cap 320 relative to the collar 340.

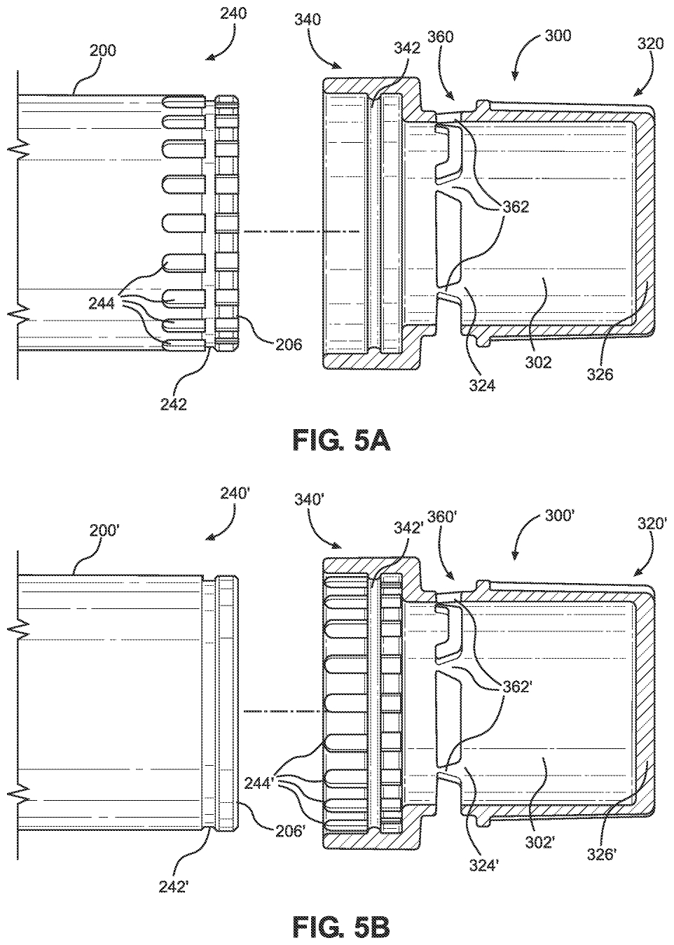

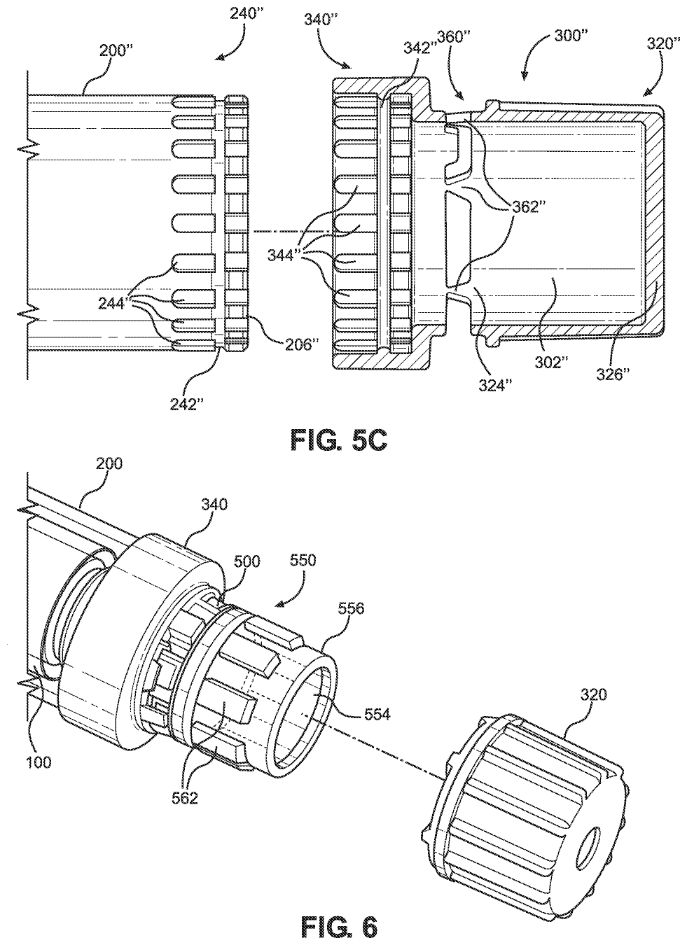

FIGS. 5A-C illustrate various embodiments of the anti-rotation feature 240 provided between the inner surface of the collar 340 and the outer surface of the barrel cover 200. The various embodiments of the anti-rotation feature 240 may prevent or reduce rotation of the collar 340 as the user rotates or twists the cap 320 to expose the distal portion of the syringe body 120 and/or tip cap 550. As illustrated in FIG. 5A, the anti-rotation feature 240 may include the plurality of teeth or grooves 244 extending longitudinally and radially outward from the distal portion of the barrel cover 200, and the inner surface of the collar 340 may have no corresponding teeth or grooves. When the collar 340 is connected around the barrel cover 200, the teeth or grooves 244 may form an interference fit within the inner surface of the collar 340 to prevent relative rotation of the collar 340 during release of the tamper evident cap 320. The teeth or grooves 244 may be longitudinally interrupted by the annular groove 242, such that two sets of teeth or grooves 244 are formed on opposing longitudinal sides of the annular groove 242. In some embodiments, as illustrated in FIG. 5B, the inner surface of the collar 340′ may include a plurality of teeth or grooves 344′, and the outer surface of the barrel cover 200′ may have no corresponding teeth or grooves. The teeth or grooves 344′ may similarly form an interference fit with the outer surface of the barrel cover 200′ to prevent relative rotation of the collar 340′ during release of the tamper evident cap 320′. The teeth or grooves 344′ may be longitudinally interrupted by the annular rib 342′, such that two sets of teeth or grooves 344′ are formed on opposing longitudinal sides of the annular rib 342′. In some embodiments, as illustrated in FIG. 5C, the anti-rotation feature 240″ may include a plurality of teeth or grooves 244″ on the outer surface of the barrel cover 200″ and a plurality of teeth or grooves 344″ on the inner surface of the collar 340″. The corresponding teeth and/or grooves 244″, 344″ may interlock to prevent rotation of the collar 340″ during rotation of the cap 320″ during breakage of the frangible connection 360″. The various embodiments of the anti-rotation feature 240, 240′, 240″ may, additionally or alternatively, include a sonic weld, adhesive, frictional surfaces, gripping material, etc. between the collar 340 and the barrel cover 200. In some embodiments, the anti-rotation feature 240 may include a film (not shown, replacing the collar 340) adhered over the barrel cover 200 to provide the frangible connection. In other embodiments, the collar 340 of the tamper evident device 300 may be integrated into the distal end of the barrel cover 200, e.g., as a singly molded component, such that an additional anti-rotation feature 240 is not needed to prevent relative rotation of the collar 340 during release of the tamper evident cap 320.

As further illustrated in FIGS. 6-7, the syringe assembly may include a tip assembly having a tip connector 500 and the tip cap 550 to seal the syringe 100 after filling. The tip connector 500 and the tip cap 550 may be received on the distal portion of the syringe 100, such that a proximal end of the tip connector 500 is received in the barrel cover 200. The tip connector 500 may extend distally out of the distal opening 206 of the barrel cover 200 to expose the tip cap 550 for removal when the syringe 100 is received in the barrel cover 200. The tip cap 550 may be engaged to a distal end of the tip connector 500 and be received in the tamper evident cap 320. In some embodiments, the syringe assembly does not contain a tip connector, and the inner surface of the tip cap 550 is configured to engage with an external surface of the syringe tip 124, e.g., by way of an interference fit (not shown).

The tip connector 500 may be a separate component and be configured to receive the syringe tip 124 during assembly. The tip connector 500 may include a tubular body having a lumen 502 configured to receive the syringe tip 124. The tip connector 500 may be made of a flexible material to allow for radial expansion under fluid pressure applied by the syringe 100. The tip connector 500 may have a plurality of ribs 504 arranged circumferentially around an outer surface. The tip connector 500 may further have internal threads (not shown) on an inner surface to engage an outer surface of the tip cap 550.

The tip cap 550 may seal the syringe tip 124 to prevent the material 20 from leaking after the chamber 130 is filled. The tip cap 550 may have a tubular body defining a lumen 552 extending through an open proximal end to a distal wall 554 of a closed distal end 556. Outer threads 558 on an outer surface of the tip cap 550 may be configured to rotationally engage the inner threads of the tip connector 500, as the lumen 552 receives the syringe tip 124. The syringe tip 124 may extend through the lumen 552 until the syringe tip 124 contacts a proximal inner surface of the distal wall 554 of the closed distal end 556 in a sealed configuration. The contact between the syringe tip 124 and the close distal end 556 may generate an audible and/or tactile indication to the user that the syringe 100 is sealed. The tip cap 550 may additionally include a radially enlarged portion 560 configured to contact a distal surface of the tip connector 500 to provide a stop for the rotation of the tip cap 550 relative to the tip connector 500. The enlarged portion 560 may extend entirely or partially around the perimeter of the tip cap 550 and may extend to the distal end 556. The tip cap 550 may include a textured surface embodied by a plurality of ribs 562 on the enlarged portion 560 to facilitate twisting and rotation of the tip cap 550 relative to the tip connector 500. Upon removal of the tamper evident cap 320 from the collar 340, the syringe 100 may remain sealed until the tip cap 550 is removed from the tip connector 500 prior to use.

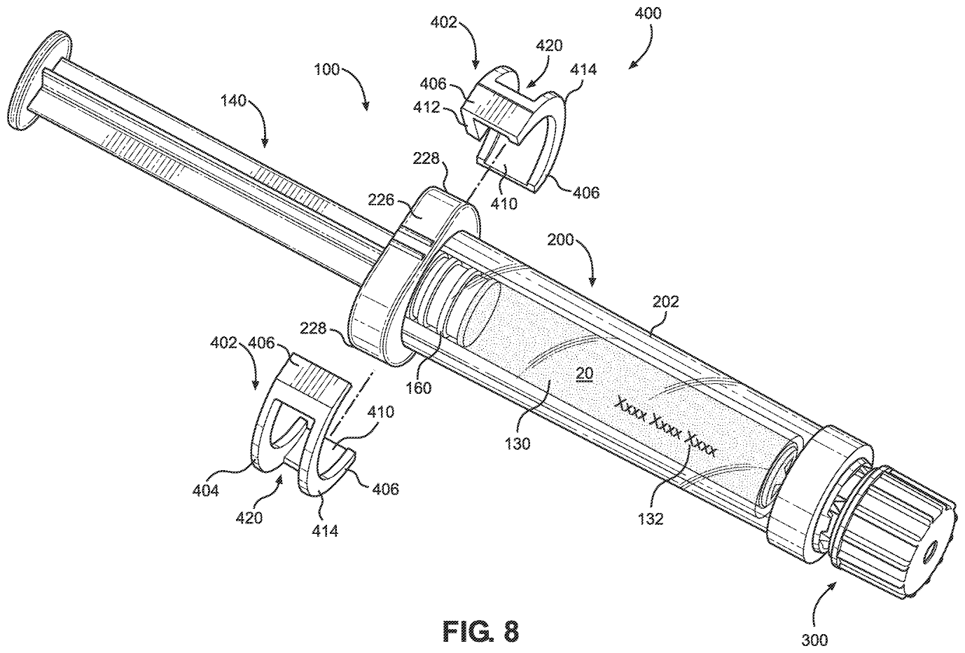

As further illustrated in FIG. 8, the backstop 400 may be releasably attached to a proximal portion of the barrel cover 200 to prevent the plunger rod 140 from being removed after the syringe 100 is filled with the material 20.

The backstop 400 may include at least two backstop members 402 releasably attachable to the proximal portion of the barrel cover 200. For example, the backstop 400 may include first and second backstop members 402, each having a slot 410 configured to snap onto the cover flange 220 and to prevent a portion of the plunger rod 140 from passing therethrough. The backstop members 402 may be separate components such that the backstop members 402 may be applied to the cover flange 220 independently. The first and second backstop members 402 may be identical, semi-circumferential pieces. The first and second backstop member 402 may be oriented 180 degrees from each other when assembled to collectively form the backstop 400. The backstop members 402 may be formed of a resiliently flexible material configured to snap onto the flange 220. For example, the cover flange 220 may include ridges and/or grooves 232 configured to engage and secure the backstop member 402. The backstop members 402 may each substantially match half of the shape of the cover flange 220. For example, the backstop members 402 may each have at least one arcuate portion 404 corresponding to the arcuate wall portion 228 of the flange 220, and at least one flat portion 406 corresponding to the substantially flat wall portions 226 of the flange 220. Although the backstop members 402 are illustrated in FIG. 8 having one arcuate portion 404 and two flat portions 406, the backstop members 402 may alternatively have one flat portion 406 and two arcuate portions 404 to fit over the cover flange 220 in a transverse configuration (not shown). In other embodiments, the backstop members 402 are semicircular. In some embodiments, the backstop members 402 may, additionally or alternatively, not be separate from each but be pivotably joined by a hinge member and/or be securable to each other on the cover flange 220 with a latch member when assembled to the cover flange 220 (not shown). The arcuate portion 404 may include an opening 420 that receives the arcuate wall portions 228 of the cover flange 220 therethrough to expose the arcuate wall portion 228 through each of the backstop members 402. Thus, the backstop 400 and the cover flange 220 may form a substantially continuous radial outer surface when the cover flange 220 is received in the backstop members 402. When assembled, the arcuate wall portions 228 of the cover flange 220 and the arcuate portion 404 of the backstop members 402 may form continuous outer arcuate surfaces, and the flat portions 406 of the backstop members 402 may join to form continuous outer flat surfaces. The continuous outer arcuate and flat surfaces may join to form a continuous perimeter surface of the flange 12 of the syringe assembly 10, as illustrated in FIG. 1. In other embodiments, the backstop members 402 do not contain an opening such that the arcuate wall portions 228 of the cover flange 220 are not exposed when the backstop 400 is assembled onto the barrel cover 200. In such embodiments, the backstop members 402 form a continuous perimeter surface of the flange 12 of the syringe assembly 10 (not shown).

The backstop 400 may prevent a portion of the plunger rod 140 (e.g., the plunger stopper 160) from moving out of the chamber 130 after filling the syringe 100 with the material 20. The backstop 400 may define a proximal opening and a distal opening having different dimensions and/or shapes. For example, the backstop members 402 may each have a proximal rim 412 collectively defining the proximal opening and a distal rim 414 collectively defining the distal opening. The slot 410 may be defined between the proximal rim 412 and the distal rim 414. The proximal rim 412 of each of the backstop members 402 may be larger or protrude further inward than the distal rim 414. Thus, the proximal opening of the backstop 400 may be smaller than the proximal opening 128 of the syringe body 120 to effectively reduce the size of the proximal opening 128 of the syringe body 120 when the backstop 400 is attached to the barrel cover 200. Therefore, the proximal rim 412 of the backstop members 402 may be configured to contact an enlarged portion of the plunger rod 140 or the plunger stopper 160 to prevent removal from the chamber 130. For example, the plunger stopper 160 may have a width larger than the reduced size of proximal opening 128 preventing removal of the plunger rod 140 proximally from the chamber 130 during shipping, handling, storage, and/or use. In some embodiments, the enlarged portion of the plunger rod 140 may be a flange that extends radially from the plunger rod body (not shown). The flange may have a substantially round cross section, but may have various other shapes depending on desired materials and manufacturing processes. In other embodiments, the enlarged portion of the plunger rod 140 may be one or more rectangular, triangular, or elliptical projections extending radially from the plunger rod body. The flange or projection may be disposed in any desired location along the length of the plunger rod 140. In some embodiments, the enlarged portion of the plunger rod 140 may be a midpoint along the length of the plunger rod 140 (not shown). In other embodiments, the enlarged portion may be at the distal end of the plunger rod 140 adjacent to the plunger stopper 160. However, the plurality of vanes 142 of the plunger rod 140 may have a width less than the enlarged portion and the proximal opening of the backstop 400 allowing passage of the plurality of vanes 142 through the proximal opening of the backstop 400, for example, during administration of the material 20. The distal rims 414 may be sized to correspond with and receive an outer surface of the barrel cover 20) when the backstop members 402 are assembled to the flange 220. The backstop 400 may also protect the syringe 100 by securing the syringe 100 to the barrel cover 200 and reducing relative movement. For example, the syringe flange 126 may be immovably secured between the ridges 230 of the cover flange 220 and the proximal rim 412 when the backstop 400 is received in the barrel cover 200. The securement may prevent damage to the syringe body 120 from relative movement with the barrel cover 200.

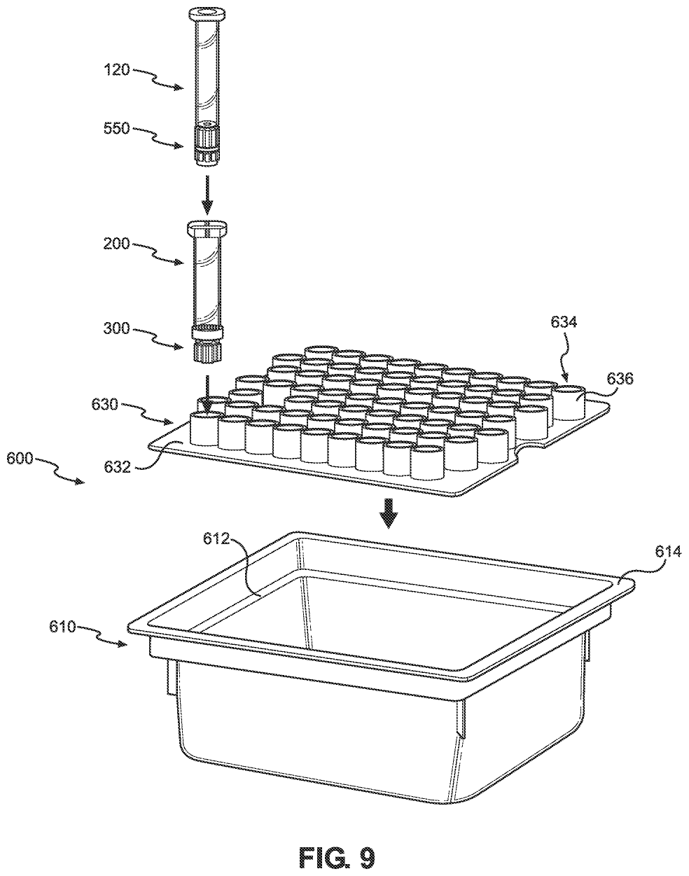

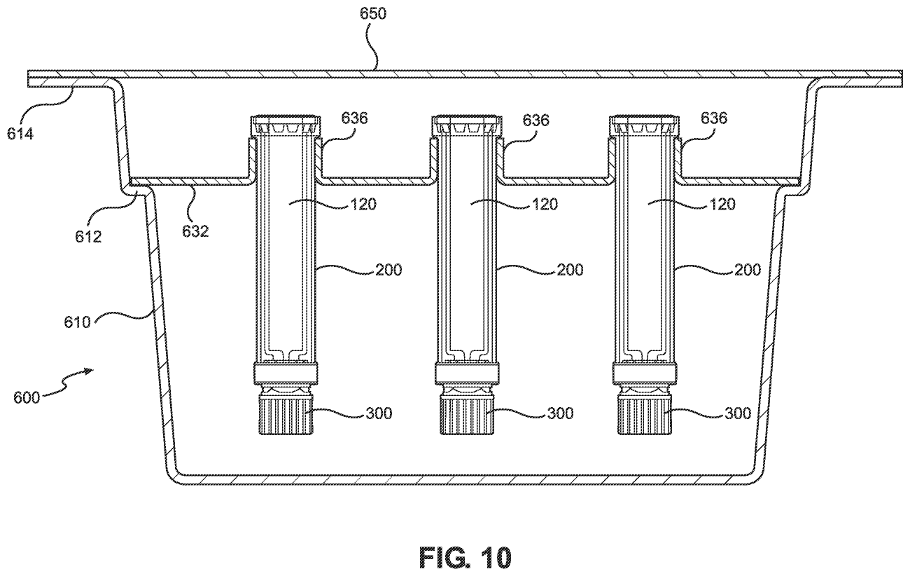

As illustrated in FIGS. 9-10, at least one syringe body 120 and barrel cover 200 may be packaged and transported in a package or container 600 prior to filling the syringe body 120 with the material 20. The container may include a tub 610 having an opening and a nest or tray 630 inserted into the opening. FIG. 10 illustrates a package of a plurality of the syringe bodies 120 each inserted into one of the barrel covers 200 in the container 600, and the container 600 is sealed.

The tray 630 may have a base 632 and a plurality of openings 634 formed by a plurality of tube members 636 extending from the base 632. As illustrated in FIGS. 9 and 10, a barrel cover 200 may extend through each of the plurality of openings 634, and a syringe body 120 may be inserted into each of the barrel covers 200. The tube members 636 may support the barrel cover flanges 220 to hold the barrel cover 200 and the syringe body 120 in an upright orientation inside of the openings 634. The shapes of the openings 634 and tube members 636 may depend upon the shape of the syringe body 120, syringe flange 126, barrel cover 200, and/or barrel cover flange 220. In some embodiments, the openings 634 are circular and the tube members 636 are cylindrical. In other embodiments, the openings 634 are oval and the tube members 636 are elliptic cylinders. In yet other embodiments, the openings 634 have two flat, oppositely positioned sides and two arcuate, oppositely positioned sides and the tube members 636 are shaped correspondingly. As further illustrated in FIG. 9, prior to insertion into the tray 630, the tip cap 550 may be attached to the syringe body 120, and/or the tamper evident device 300 may be attached to the barrel cover 200. The barrel cover 200 being in the tray 630 to receive the syringe body 120 prior to filling with the material 20 may increase assembly efficiency by reducing processing time and steps needed to be performed after filling of the syringe body 120. Pre-assembly of the tamper evidence device 300 to the barrel cover 200 prior to insertion into the tray 630 and/or to filling the syringe body 120 with the material 20 may further increase assembly efficiency by reducing processing time and steps needed to be performed after filling of the syringe body 120. The barrel cover flange 220 may rest on an upper surface of the tube members 636 when inserted, and the ridges 230 may support the syringe flange 126 to provide stability and an improved seat for the syringe flange 126. The tube members 636 may hinder the barrel cover 200 from swinging and contacting each other when supported by the tray 630. The barrel cover 200 may further reduce any damage to the syringe body 120 when the barrel covers 200 contact each other during shipping and handling. The base 632 may be substantially planar and extend around the plurality of tube members 636. The base 632 may be supported by an inner shoulder 612 of the tub 610 when the tray 630 is inserted into the tub 610.

As further illustrated in FIG. 10, the package or container 600 may be sealed by a film or sheet 650 after insertion of the tray 630 with the barrel covers 200, tamper evident devices 300 and empty syringe bodies 120 into the tub 610. A peripheral edge of the sheet 650 may be attached to a rim 614 of the tub 610 with an adhesive to seal the interior of the tub 610. The sheet 650 may be selectively impervious. The sheet 650 may block any contaminating particles, including contamination by microorganisms, bacteria and/or a biologically active material which may come into contact with the container 600 when handled. The sheet 650 may be permeable to a sterilizing gas or fluid to enable sterilization of the empty syringe bodies 120, the barrel covers 200, and/or the tamper evident devices 300 while sealed in the tub 610. The adhesive may be solvent-free, such as water-based or hot-melt adhesives. The adhesive may be peelable and not generate particles or fibers when the sheet 650 is detached from the tub 610.

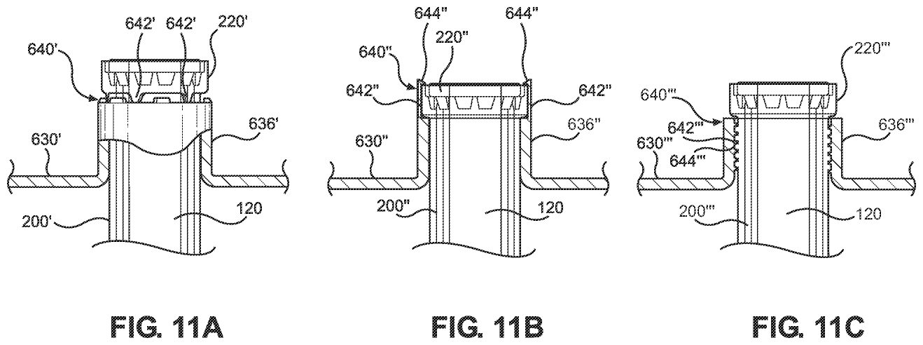

In some embodiments, the barrel cover 200 may be releasably attached and/or secured to the tube members 636 of the tray 630 by one or more of a friction fit, a press fit, a snap fit, a rotational connection, and/or a frangible connection, as illustrated in FIGS. 11A-C. A force sufficient for detaching by pulling the barrel covers 200 relative to the tray 630 may be greater than a gravitational force, such that the barrel covers 200 do not readily fall out for example when the tray 630 is turned upside down. However, the syringe body 120 may be freely supported within the barrel cover 200 during initial packaging and prior to filling by resting the syringe flange 126 onto the cover flange 220 through a gravitational force, as discussed herein.

For example, as illustrated in FIG. 11A, the barrel cover 200′ may be releasably attached and/or secured to the tray 630′ at a frangible connection 640′. The tray 630′ may be formed as a unitary, one-piece body with the barrel cover 200′ and/or the tamper evident device 300 through injection molded. For example, the frangible connection 640′ may be initially formed between the barrel cover 200′ and the tray 630′ through the injection molding process, such that the barrel cover 200′ and enclosed syringe body 120 may be removed from the tray 630′ by the final user (or during intermediate processing) by breaking the frangible connections 640′ through twisting and/or pulling the barrel cover 200′ relative to the tray 630′. As illustrated, the frangible connection 640′ may be formed by bridges 642′ circumferentially disposed around the opening 634 and connecting the cover flange 220′ and the tube members 636′. The bridges 642′ may be broken by a user, similar to the frangible connection 360 as discussed above.

As illustrated in FIG. 11B, the barrel cover 200″ may be releasably attached and/or secured to the tray 630″ by a mating and/or snap fit connection 640″. As illustrated, the mating and/or snap fit connection 640″ may be formed by pivoting arms 642″ extending from the top of the tube members 636″, where each of the pivoting arms 642″ has a protrusion 644″ at a free end extending radially inwardly. The tube members 636″ may have a pair of pivoting arms 642″ on opposite sides of the narrower width of the cover flange 220″ (e.g., corresponding to width of arcuate wall portions 228). The cover flange 220″ may be configured to deflect the pivoting arms 642″ radially outwardly by camming with the protrusions 644″ when inserted, and the pivoting arms 642″ may pivot back for the protrusions 644″ to releasably secure the upper surface of the cover flange 220″. The mating and/or snap fit connection 640″ may secure the barrel cover 200″ to the tray 630″ until the protrusions 644″ are released with application of a sufficient force to push, pull and/or twist the barrel cover 200″ relative to the tray 630″. The protrusions 644″ may have upper and/or lower camming surfaces to facilitate insertion and/or removal of the barrel covers 200″ by the user. The mating and/or snap fit connection 640″ may additionally form alignment features that retain the barrel covers in a predetermined orientation, such as the cover flanges 220″ being retained parallel to each other to optimize space. Additionally or alternatively, the barrel cover 200″ may have an outer diameter that approximates the inner diameter of the openings 634 (e.g., the tube members 636″), such that the barrel cover 200″ is retained in the openings 634 through a friction fit until a sufficient force is applied to pull and/or twist the barrel cover 200 relative to the tray 630.

As illustrated in FIG. 11C, the barrel cover 200′″ may be releasably attached to the tray 630′″ by a rotational connection 640′″. Thus, barrel cover 200′″ may be inserted and/or released from the tube member 636′″ through relative rotation. For example, the rotational connection 640′″ may include a thread 642′″ on an outer surface of the barrel cover 200′″ and a thread 644′″ on an inner surface of the tube members 636′″ configured to rotationally engage each other. However, the rotational connection 640′″ may alternatively be attached through other rotational connections such as a lug on an outer surface of the barrel cover 200′″ that is rotationally received in a groove on the tube member 636′″. Thus, the barrel cover 200′″ and enclosed syringe body 120 may be connected and/or released from the tray 630′″ through at least a quarter relative rotation around the longitudinal axis of the tube member 636′″.

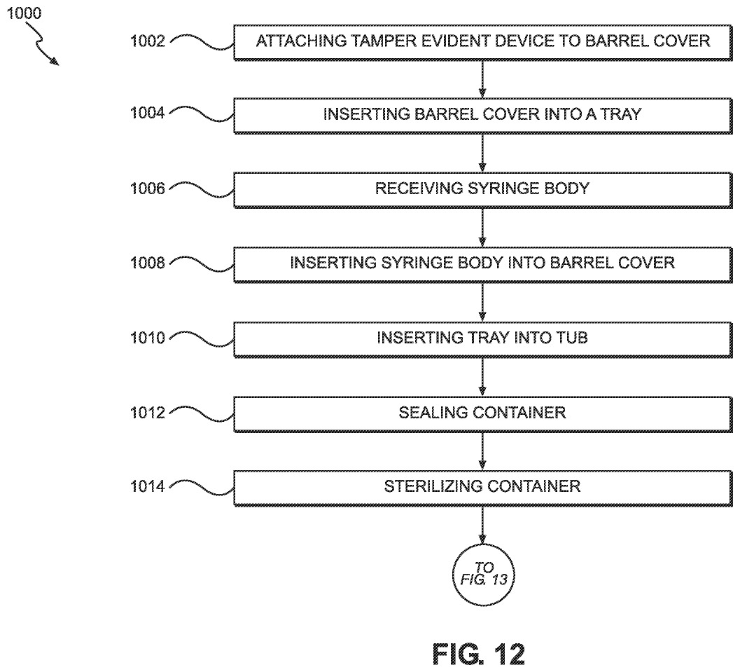

FIG. 12 illustrates a method 1000 of packaging the syringe bodies 120. The method 1000 may begin with step 1002, where the tamper evident device 300 may be attached to the distal portion of the barrel cover 200. The annular rib 342 on the collar 340 may be receive in the annular groove 242 of the barrel cover 200. However, in some embodiments, the barrel cover 200 and tamper evident device 300 are provided as a pre-assembled or integral component. For example, the barrel cover 200 and the tamper evident device 300 may be provided as a single molded component (e.g., injection molded). In step 1004, the barrel cover 200 may be inserted into the tray 630. As illustrated in FIGS. 9-10, the barrel cover 200 may be inserted into one of the openings 634 defined by the tube members 636, such that the cover flange 220 rests on an upper surface of the tube members 636 when inserted. As discussed above, in some embodiments, the barrel cover 200 may additionally or alternatively be releasably attached and/or secured to the tray 630 via a friction fit, a press fit, a snap fit, a rotational connection, and/or a frangible connection. For example, in some embodiments, the tray 630 may be formed as an integral, unitary body through injection molding with the barrel cover 200 and/or the tamper evident device 300. In step 1006, the syringe body 120 may be received, where the syringe body 120 has the syringe tip 124 and the proximal opening 128. As discussed above, the syringe body 120 may define a chamber 130 extending along the longitudinal direction between the syringe tip 124 and the proximal opening 128. The tip cap 550 may be pre-assembled to the syringe body 120, or the tip cap 550 may be attached to the syringe body 120, as discussed above. In step 1008, the syringe body 120 may be inserted into the proximal opening 204 of the barrel cover 200, such that the syringe flange 126 is received in the cover flange 220, and the tip cap 550 extends out of a distal opening of the barrel cover 200 and is received in the cap 320. It is contemplated that step 1006 of inserting the syringe body 120 into the barrel cover 200 may be performed before or after step 1004 of inserting the barrel cover 200 into the tray 630. It should also be recognized that steps 1002-1008 may be repeated for how many syringe bodies 120 are to be inserted into the container 600, each of the syringe bodies 120 inserted into one of a plurality of the barrel covers 200 and/or a plurality of tamper evident devices 300 each attached to one of the barrel covers 200. In step 1010, the tray 630 may be inserted into the tub 610. Step 1010 may be performed before or after inserting the barrel cover 200 and/or the syringe body 120 into the tray 630. In step 1012, the container 600 may then be sealed by attaching the sheet 650 to the rim 614 of the tub 610 enclosing the empty syringe bodies 120 in the barrel covers 200, as illustrated in FIG. 10. In step 1014, the sealed package or container 600 with the empty syringe bodies 120 may be sterilized to ensure sterility of the chamber 130 prior to filling. For example, the sealed package or container 600 and the empty syringe bodies 120 may be sterilized through exposure to a high-pressure saturated steam in an autoclave machine. The sealed package or container 600 may also be transported to a filling station, before or after the sterilizing step.

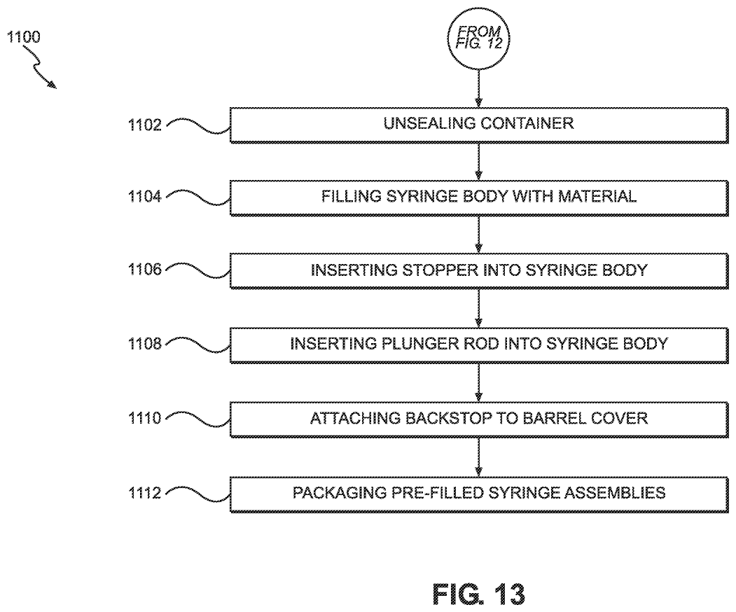

FIG. 13 illustrates a method 1100 of filling the syringe bodies 120 and packaging the syringe assemblies 10 as prefilled syringe assemblies. In step 1102, the package or container 600 may be unsealed by removing the sheet 650 from the tub 610 at a filling station to expose the proximal openings 128 of the syringe bodies 120. In step 1104, the syringe body 120 may be filled with the material 20. For example, the tray 630 may be removed together with the syringe bodies 120 and barrel covers 200 from the tub 610 and transported to a filling machine (not shown). The plurality of syringe bodies 120 may be filled with the material 20 simultaneously by dispensing the material 20 from a plurality of filling nozzles of the filling machine into the exposed proximal openings 128 of each of the syringe bodies 120. In step 1106, the plunger stopper 160 may then be inserted into each of the syringe bodies 120 to seal the proximal opening 128 of each of the syringe bodies 120. In step 1108, the plunger rod 140 may then be inserted into each of the chambers 130 and attached to a proximal end of the plunger stopper 160. For example, the distal end of the plunger rod 140 may be attached to the plunger stopper 160 through a threaded fit, a snap fit, an interference fit, and/or a magnetic attachment. It is also contemplated that steps 1106 and 1108 may be performed together with a pre-assembled plunger rod 140 and plunger stopper 160. In step 1110, the backstop 400 may be attached to the proximal portion of each of the barrel cover 200. For example, the backstop 400 may include first and second backstop members 402 configured to snap on opposing sides of the cover flange 220, as discussed above and illustrated in FIG. 8. The backstop members 402 may include a proximal rim 412 that effectively narrows the proximal opening 128 of the syringe 100, preventing withdrawal of the portion of the plunger rod 140 from the chamber 130. In some embodiments, step 1002 of attaching the tamper evident device may be performed after step 1108 of inserting the syringe body 120 into the proximal opening 204 of the barrel cover 200 or after the step 1110 of attaching the backstop 400 to the proximal portion of the barrel cover. However, attaching the tamper evident device 300 prior to filling increases efficiency downstream. In step 1112, the syringe assemblies 10 may be packaged as prefilled syringe assemblies for shipment from the filling station to a user. For example, the tray 630 with the syringe assemblies 10 may be reinserted into the same or a different tub 610 and resealed with the same or a different sheet 650. Alternatively, the syringe assemblies 10 may be packaged in a different form of packaging individually, in pairs, or in any other types or combinations or arrangements. In some embodiments of the method 1100, the barrel covers 200 may be detached from the tray 630 by removing the connection 640 (e.g., the friction fit, the press fit, the snap fit, the rotational connection, and/or the frangible connection), for example prior to the plunger rod 140 being inserted in step 1108 and/or prior to the backstop 400 being attached in step 1110. The barrel covers 200 may be reattached to the tray 630 by the connection 640 prior to step 1112.

While systems and methods have been described in connection with the various embodiments of the various figures, it will be appreciated by those skilled in the art that changes could be made to the embodiments without departing from the broad inventive concept thereof. It is understood, therefore, that this disclosure is not limited to the particular embodiments disclosed, and it is intended to cover modifications within the spirit and scope of the present disclosure as defined by the claims.

What is claimed is:

1. A package comprising:
a tray having a plurality of openings; and
a plurality of barrel covers,
wherein each of the plurality of barrel covers extend through one of the plurality of openings.

2. The package of claim 1, further comprising a plurality of syringe bodies, wherein each of the syringe bodies is inserted into one of the plurality of barrel covers through a corresponding opening.

3. The package of claim 2, further comprising a plurality of syringe caps, each of the plurality of syringe caps configured to seal a distal portion of one of the plurality of syringe bodies when the plurality of syringe bodies are inserted into the plurality of barrel covers through the plurality of openings.

4. The package of claim 2, wherein each of the plurality of syringe bodies includes a flange at a proximal portion of the syringe body, and each of the plurality of barrel covers includes a plurality of ridges supporting the flange of one of the plurality of syringe bodies.

5. The package of claim 2, wherein the syringe bodies are empty.

6. The package of claim 2, wherein the syringe bodies are made of glass.

7. The package of claim 2, wherein each of the plurality of barrel covers has a proximal opening and a distal opening, and each of the plurality of syringe bodies extends distal of the distal opening of each of the plurality of barrel covers.

8. The package of claim 1, further comprising a plurality of tamper evident devices, each of the plurality of tamper evident devices including a collar, a tamper evident cap, and a frangible connection between the collar and the tamper evident cap, wherein each collar of each of the plurality of tamper evident devices is attached to one of the plurality of barrel covers.

9. The package of claim 1, wherein each of the plurality of barrel covers is translucent or transparent.

10. The package of claim 1, wherein the tray has a plurality of tube members extending from an upper surface of the tray, each of the plurality of tube members defining one of the plurality of openings, and each of the plurality of barrel covers being supported by one of the plurality of tube members.

11. The package of claim 1, further comprising a tub including a shoulder portion configured to support the tray.

12. The package of claim 11, further comprising a sheet adhered to a rim of the tub and covering the tray, wherein the sheet is permeable to sterilizing gas and/or vapor.

13. The package of claim 1, wherein the plurality of barrel covers are releasably attached to the tray by at least one of a friction fit, a press fit, a snap fit, a rotational connection, or a frangible connection.

14. A package comprising:
a tray having a plurality of tube members extending from an upper surface, each of the plurality of tube members defining an opening;
a tub receiving the tray;
a plurality of barrel covers extending through the openings of the plurality of tube members;
a plurality of syringe bodies in the plurality of barrel covers; and
a plurality of tamper evident devices attached to the plurality of barrel covers,
wherein each tamper evident device includes a collar, a tamper evident cap, and a frangible connection between the collar and the tamper evident cap.

15. The package of claim 14, wherein the plurality of barrel covers are releasably attached to the tray by at least one of a friction fit, a press fit, a snap fit, a rotational connection, or a frangible connection.

16. A method of packaging a plurality of syringe bodies, the method comprising:
inserting the plurality of syringe bodies into a plurality of barrel covers positioned in a tray; and
inserting the tray into a tub.

17. The method of claim 16, further comprising inserting the plurality of barrel covers into a plurality of openings of the tray.

18. The method of claim 16, further comprising sealing the tub by attaching a sheet to an upper flange of the tub, wherein the sheet is permeable to sterilizing gas and/or vapor.

19. The method of claim 16, wherein the plurality of syringe bodies are empty when inserted into the barrel covers.

20. The method of claim 16, further comprising releasably attaching the plurality of barrel covers to the tray via at least one of a friction fit, a press fit, a snap fit, a rotational connection, or a frangible connection.
